(19) United States
(12) Patent Application Publication
(10) Pub. No.: US 2023/0215571 A1
(43) Pub. Date: Jul. 6, 2023

(54) AUTOMATED CLASSIFICATION OF IMMUNOPHENOTYPES REPRESENTED IN FLOW CYTOMETRY DATA

(71) Applicant: AHEAD Intelligence Ltd., Taipei City (TW)

(72) Inventors: Yu-Fen Wang, Taipei City (TW); Chang-Hsing Liang, Taipei City (TW); Chi-Chun Lee, Taipei City (TW); Jeng-Lin Li, Taipei City (TW); Wen-Chieh Sung, Taipei City (TW); Yu-Lin Chen, Taipei City (TW)

(21) Appl. No.: 18/182,798

(22) Filed: Mar. 13, 2023

Related U.S. Application Data
(63) Continuation of application No. PCT/US2021/050301, filed on Sep. 14, 2021.
(60) Provisional application No. 63/078,312, filed on Sep. 14, 2020.
(60) Provisional application No. 63/078,662, filed on Sep. 15, 2020.

(51) Int. Cl. G16H 50/20; G01N 15/14

(52) U.S. Cl. CPC G16H 50/20; G01N 15/1459; G01N 15/1429; G01N 2015/1006

(57) ABSTRACT

Introduced here is an approach to improving the automatic identification of hematological diseases using computer-implemented models that are trained to rapidly distinguish between different collections of immunophenotypes that represent different disease types or disease states. Understanding the different patterns of immunophenotype collections contained in a given sample may permit a proposed diagnosis for a given hematological disease to be produced for the corresponding patient. For example, the proposed diagnoses may be output by a classification model based on the distribution of immunophenotypes across the given sample.

### CROSS-REFERENCE TO RELATED APPLICATIONS

This application is a continuation of International Patent Application No. PCT/US2021/050301, titled “AUTOMATED CLASSIFICATION OF IMMUNOPHENOTYPES REPRESENTED IN FLOW CYTOMETRY DATA” and filed Sep. 14, 2021, which claims priority to U.S. Provisional Application No. 63/078,312, titled “Systems and Methods for Automatic Classification of Flow Cytometry Data” and filed on Sep. 14, 2020, and U.S. Provisional Application No. 63/078,662, titled “Methods for Automatic Preprocessing Flow Cytometry Data” and filed on Sep. 15, 2020, each of which are incorporated by reference herein in their entireties.

### TECHNICAL FIELD

Various embodiments concern computer programs and associated computer-implemented techniques for classifying flow cytometry data in an automated manner.

### BACKGROUND

Leukemia (occasionally spelled “leukaemia”) are hematological diseases that start in cells that would normally develop into different types of blood cells. Often, leukemias begin in the bone marrow and result in high numbers of abnormal blood cells. These abnormal blood cells may be referred as “leukemia cells” or “blast cells.” The exact cause of leukemia is unknown, so a diagnosis is normally made based on the results of a blood test or bone marrow test (also referred to as a “bone marrow biopsy”). Generally, the blood test or bone marrow biopsy is taken when an individual (also referred to as a “patient” or “subject”) reports that she is suffering from symptoms such as bleeding, bruising, fatigue, and fever.

There are four main types of leukemia—acute lymphoblastic leukemia (ALL), acute myeloid leukemia (AML), chronic lymphocytic leukemia (CLL), and chronic myeloid leukemia (CML)—as well as a number of less common types. Leukemias belong to a broader group of conditions that affect the blood, bone marrow, and lymphoid system. This broader group of conditions are commonly referred to as “tumors of the hematopoietic and lymphoid tissues.”

The aforementioned types have historically been divided based mainly on (i) whether the leukemia is acute (i.e., fast growing) or chronic (i.e., slow growing) and (ii) whether the leukemia starts in myeloid cells or lymphoid cells. ALL and AML generally start in the bone marrow but then often move into the blood and other parts of the human body, including the lymph nodes, liver, and spleen. The rate at which blast cells (or simply “blasts”) spread through the human body corresponds to whether the underlying leukemia is acute or chronic. The presence and prevalence of blast cells can also be indicative of other hematological diseases, such as lymphoma and multiple myeloma.

### BRIEF DESCRIPTION OF THE DRAWINGS

FIG. 1 includes a chart that illustrates how hematological diseases have historically been classified.

FIG. 2A includes a high-level illustration of a framework that can be implemented by an analysis platform to acquire, process, and transform flow cytometry (FC) data to facilitate automated detection of hematological abnormalities that are indicative of hematological diseases.

FIG. 2B illustrates how the framework shown in FIG. 2A can be used to (i) acquire “raw” FC data that is associated with a patient, (ii) select intersecting or interrelating parameters, (iii) transform the “raw” FC data through patient-level encoding, and then either (iv) classify the patient by applying a classification model to the transformed FC data or (v) train a classification model to do the same.

FIG. 3 includes a high-level illustration of a process by which FC data is obtained from a source.

FIG. 4 illustrates how the spillover signal from other fluorescence intensities can bias the pure signal of the primary fluorescence intensity that is presently of interest.

FIG. 5 illustrates how a scatter plot can be generated with forward scatter height (FSC-H) along the y-axis and forward scatter area (FSC-A) along the x-axis to facilitate manual singlets gating.

FIG. 6 includes a flow diagram of a process for automatically performing singlet gating.

FIG. 7 includes a flow diagram of a process for normalizing an FC dataset that is extracted from a Flow Cytometry Standard (FCS) file.

FIG. 8 includes a high-level illustration of a process by which processed FC data is transformed from its matrix form into a vector.

FIG. 9 includes a flow diagram of a process for training a model to classify hematological diseases.

FIG. 10 includes a flow diagram of a process for classifying a sample through the application of a classification model.

FIG. 11 illustrates a network environment that includes an analysis platform.

FIG. 12 includes a diagram illustrating one example of a system that is able to automatically classify different patterns of immunophenotype collections so as to identify hematological diseases.

FIG. 13 is a block diagram illustrating an example of a processing system in which at least some operations described herein can be implemented.

Various features of the technology described herein will become clearer to those skilled in the art by studying the Detailed Description in conjunction with the drawings. Certain embodiments are shown in the drawings for the purpose of illustration. However, those skilled in the art will recognize that alternative embodiments may be employed without departing from the principles of the technology. Accordingly, although specific forms of the technology are shown in the drawings, the technology is amenable to various modifications.

### DETAILED DESCRIPTION

To understand leukemia and lymphoma, it helps to understand the blood and lymph systems of the human body.

Bone marrow is the soft inner part of some bones. At a high level, bone marrow is comprised of blood-forming cells, fat cells, and supporting tissues. A small fraction of the blood-forming cells in the bone marrow are normally blood stem cells. Inside the bone marrow, blood stem cells undergo changes in order to develop into red blood cells, platelets, or white blood cells. Red blood cells (RBCs) carry oxygen from the lungs to other tissues into the human body, as well as take carbon dioxide back to the lungs for removal (e.g., via exhalation). Platelets are cell fragments that are made from a type of blood stem cell called a “megakaryocyte.” Platelets are important in plugging holes in blood vessels that are caused by cuts, bruises, and the like. White blood cells (WBCs) are responsible for helping the human body fight off infections.

There are three main types of WBCs—lymphocytes, granulocytes, and monocytes. Lymphocytes are the main cells that make up the lymph tissue found in lymph nodes and other parts of the human body. Lymphocytes develop from calls called “lymphoblasts” to become mature, infection-fighting cells. There are two main types of lymphocytes—B lymphocytes (also referred to as “B cells”) and T lymphocytes (also referred to as “T cells”). B cells help protect the human body by making proteins called antibodies that attach to germs, while T cells generally help destroy those germs. ALL develops from early forms of lymphocytes. ALL can start in early B cells or T cells at early stages of maturity. Lymphoma also starts in the lymphocytes, though it normally affects B cells or T cells in the lymph nodes rather than the blood and bone marrow. Granulocytes are WBCs that contain granules. These granules normally contain enzymes and other substances that may be helpful in destroying germs. There are three types of granulocytes—neutrophils, basophils, and eosinophils—that can be distinguished by the size and color of the granules. Monocytes also help protect the body against bacteria. Normally, monocytes circulate in the bloodstream for a relatively short interval of time (e.g., roughly one day) and then enter the tissues to become macrophages, which can destroy germs by surrounding and then digesting them.

The term “myeloid cell” is normally used to refer to those blood stem cells that can develop into RBCs, platelets, or WBCs other than lymphocytes. In contrast to ALL, these myeloid cells are the ones that are abnormal in the case of AML.

The lymphatic system (also referred to as the “lymphoid system”) is an organ system that is part of the circulatory system and immune system. The lymphoid system is made up of a large network of lymph, lymphatic vessels, lymph nodes, lymphatic organs, and lymphatic tissues. The vessels carry a clear fluid referred to as “lymph” towards the heart. Unlike the cardiovascular system, the lymphatic system is not a closed system. This means that problems affecting the lymphoid system can quickly spread throughout the body without timely treatment.

As mentioned above, leukemia diagnoses are normally made by healthcare professionals based on the results of blood tests or bone marrow tests. By looking at a sample of the blood of an individual, a healthcare professional can determine whether there are abnormal levels of RBCs, platelets, or WBCs—which may suggest leukemia. A blood test could also show the presence of blasts, though not all types of leukemia cause blasts to circulate in the blood. Sometimes blasts stay in the bone marrow. For that reason, the healthcare professional may recommend a bone marrow test in which a sample of the bone marrow is removed in order to look for blasts, or the healthcare professional may recommend a spinal fluid test in which a sample of the cerebrospinal fluid is removed in order to look for blasts.

While recent advances in medicine have improved the survival rates of individuals diagnosed with leukemia, unexpected outcomes still abruptly affect the prognosis in some cases. Current clinical practice uses the identification of minimal residual disease (MRD) as a prognosis indicator that is detected using flow cytometry (FC). At a high level, immunotyping by FC is a laboratory technique that is used to measure physical and chemical characteristics of a population of cells.

In an FC experiment (or simply “experiment”), a sample containing cells is initially suspended in a fluid. Normally, these cells are labeled with fluorescent markers that only bind to certain types of cells, so as to define different types of cells. The sample is then injected into a flow cytometer instrument (or simply “flow cytometer”), where the sample is focused—ideally one cell at a time—through a laser beam. The light scattered by the cells is characteristic to the cells, thereby creating illumination patterns that reflect cell types contained in the sample. Because the cells are labeled with fluorescent markers, light will be absorbed and then emitted within specific bands of wavelengths.

Accordingly, the experiment may involve measuring fluorescent excitement on antibody markers to produce FC data. Historically, healthcare professionals have manually examined FC data through visual analysis of two-dimensional plots in order to determine appropriate diagnoses. This approach is not only laborious and time-consuming since the number of cells tends to range from tens of thousands to millions, but also prone to error since these healthcare professionals must make subjective decisions. Several entities have proposed adapting machine learning (ML) algorithms or artificial intelligence (AI) algorithms for managing FC data; however, handling massive amounts of FC data remains a challenge.

Introduced here is an approach to improving the automatic identification of hematological diseases using computer-implemented models (or simply “models”) that are trained to rapidly distinguish between different collections of immunophenotypes that represent different disease types or disease states. Understanding the different patterns of immunophenotype collections contained in a given sample may permit a proposed diagnosis for a given hematological disease to be produced for the corresponding patient. The proposed diagnosis may be one of multiple outputs that are produced by a disease analysis platform (or simply “analysis platform”) based on the distribution of immunophenotypes across the sample. For example, the analysis platform may be able to produce proposed diagnoses for more than one type of acute leukemia (e.g., ALL and AML), pancytopenia (e.g., bone marrow neoplasia and one or more non-neoplastic conditions), or another kind of hematological disease.

This approach can be employed as part of a training framework for training a model to automatically classify a sample that is represented by FC data. The training framework may have three steps, namely, a first step in which FC data is processed, a second step in which the processed FC data is transformed into a format that is better suited for training a model, and a third step in which the formatted and processed FC data is used to train the model. Generally, the training framework is implemented tens, hundreds, or thousands of times since various samples (e.g., corresponding to different hematological diseases) can be used for training. This paradigm (i.e., processing, transforming, and then training) allows insights into the distribution of cell types across a sample to be reliably and rapidly obtained and then presented, for example, to healthcare professionals for consideration.

This approach can also be employed as part of a classifying framework for applying a trained model to FC data to produce one or more outputs. Each output may be representative of a proposed diagnosis for a hematological disease. At a high level, the classifying framework may be similar to the training framework as the processing and transforming steps may also be performed. Accordingly, upon receiving input indicative of a request to produce proposed diagnoses for a sample based on an analysis of FC data, the FC data can initially be processed and then transformed into the format that can be more easily handled by the trained model. Then, the formatted and processed FC data can be provided to the trained model, as input, in order to produce the output(s). In contrast with manual analysis of FC data to classify cell types, this automated approach can improve the quality, consistency, and timeliness of health care by rapidly surfacing insights that can be used for diagnosing and monitoring patients.

While embodiments may be described with reference to particular hematological diseases, these hematological diseases were selected for the purpose of illustration. As an example, the approach may be described in the context of a model that, when applied to FC data corresponding to a sample, is able to produce outputs indicative of proposed diagnoses for ALL, AML, and pancytopenia. However, the approach may be similarly applicable to other hematological diseases, such as CLL, CML, Hodgkin lymphoma and non-Hodgkin lymphoma (diffuse large B-cell lymphoma, follicular lymphoma, mantle cell lymphoma, T-cell lymphoma), multiple myeloma, acute erythroid leukemia (AEL), acute promyelocytic leukemia (APL), and other solid tumors. Moreover, the approach may be similarly applicable to malignant hematological diseases and non-malignant hematological diseases (e.g., pancytopenia). Accordingly, the model may be able to stratify a patient amongst various hematological diseases—malignant and/or non-malignant—based on a sample-level representation of cells discovered in the sample.

Embodiments may also be described in the context of executable instructions for the purpose of illustration. However, those skilled in the art will recognize that aspects of the present application could be implemented via hardware, firmware, or software. As an example, an analysis platform could be embodied as a computer program that offers support for reviewing information related to the progression and/or status of a hematological disease, cataloging treatments, reviewing diagnoses proposed by models, and the like.

### Terminology

References in the present disclosure to “an embodiment” or “some embodiments” mean that the feature being described is included in at least one embodiment. Occurrences of such phrases do not necessarily refer to the same embodiment, nor are they necessarily referring to alternative embodiments that are mutually exclusive of one another.

Unless the context clearly requires otherwise, the terms “comprise,” “comprising,” and “comprised of” are to be construed in an inclusive sense rather than an exclusive or exhaustive sense (i.e., in the sense of “including but not limited to”). The term “based on” is also to be construed in an inclusive sense rather than an exclusive or exhaustive sense. Thus, unless otherwise noted, the term “based on” is intended to mean “based at least in part on.”

The terms “connected,” “coupled,” and variants thereof are intended to include any connection or coupling between two or more elements, either direct or indirect. The connection or coupling can be physical, logical, or a combination thereof. For example, elements may be electrically or communicatively coupled to one another despite not sharing a physical connection.

The term “module” may refer broadly to software, firmware, hardware, or combinations thereof. Modules are typically functional components that generate one or more outputs based on one or more inputs. For example, a computer program may include or utilize multiple modules that are responsible for completing different tasks, or a computer program may include or utilize a single module that is responsible for completing all tasks.

When used in reference to a list of multiple items, the term “or” is intended to cover all of the following interpretations: any of the items in the list, all of the items in the list, and any combination of items in the list.

### Introduction to Immunophenotyping

Immunophenotyping by FC is a laboratory technique that is generally used to detect the presence or absence of WBC markers called antigens. These antigens are protein structures that are found on or in WBCs, and specific groupings of these antigens are unique to specific cell types. Because FC immunophenotyping can serve as a sensitive screen for hematological diseases, it is a useful tool for staging previously diagnosed hematological diseases, demonstrating the absence of hematological diseases, monitoring responses to treatment (e.g., through analysis of MRD), documenting relapse or progression of hematological diseases, and detecting intercurrent hematological diseases. Simply put, FC immunophenotyping can be used to detect normal cells in addition to abnormal cells whose pattern of markers are generally observed with specific hematological diseases.

Traditionally, the FC data generated by flow cytometers has been either plotted in a single dimension to produce a histogram or plotted in multiple dimensions to product a “dot plot” or “scatter plot.” The regions on these plots are sequentially separated based on fluorescence intensity by creating a series of subset extractions (also referred to as “gates”). Specific gating protocols exist for diagnostic purposes, especially in relation to hematology. Single cells have historically been distinguished from doublets and higher aggregates through visual analysis of these plots. The term “doublet,” as used herein, may refer to an event where more than one cell is measured by a flow cytometer. Doubles are normally identified based on the “time-of-flight” or “pulse-width” through the laser beam. Properly identifying doublets is critical in cell sorting since the corresponding values in the FC data should not impact the analysis. However, because doublet exclusion relies heavily on visual analysis, the process is prone to errors as further discussed below.

Using FC data, an individual can determine the relative size of cells using a known control. For example, forward scatter (FSC) and side scatter (SSC) values are commonly used in gating. More specifically, FSC and SSC values can be used to identify cells of interest based on size and granularity. Generally, FSC and SSC values are used to standardize data that is related to other light scatter parameters, especially the fluorescent markers used to identify the different cell types through traditional visual analysis of FC data.

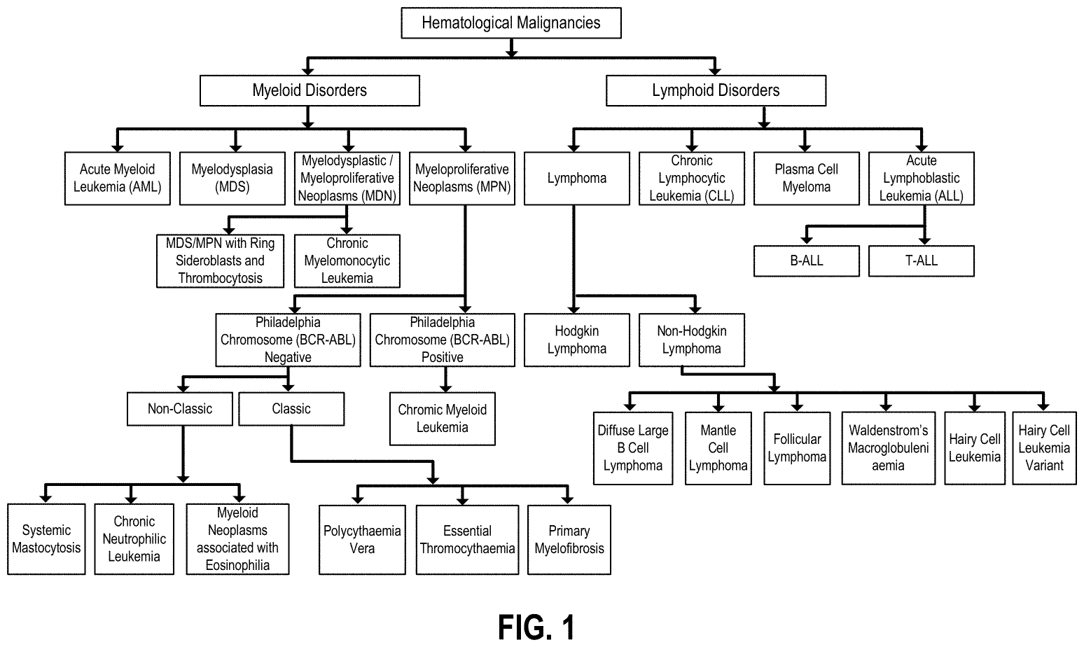

There are several drawbacks to FC immunophenotyping, however. First, data that is generated by a flow cytometer over the course of an experiment can be difficult to comprehend. This can lead to errors since the healthcare professionals responsible for analyzing the data may need to make subjective decisions. Second, determining an appropriate hematological disease classification can be difficult. FIG. 1 includes a chart that illustrates how hematological diseases have historically been classified. Properly navigating this chart relies on an accurate understanding of the distribution of cell types in a sample, however. Cells can easily be mischaracterized due to the limitations of visual analysis of FC data populated on plots.

The approach introduced here not only involves classifying individual cells in an automated manner to reduce errors, but may also involve generating representations of cell types across samples in order to determine how to stratify the corresponding patients among different hematological diseases. Said another way, a sample-level representation (also referred to as a “patient-level representation”) of cell types may be used to determine which hematological disease, if any, to predict for a given sample (and thus a given patient). Sample-level representations could be helpful in classifying patients among different hematological diseases, as well as assigning a pathological status (e.g., relapse, progression, etc.) and correlating cell type distribution to clinical intervention to establish efficacy. Examples of clinical interventions include chemotherapy, target therapy, immune checkpoint inhibitors, and chimeric antigen receptor (CAR) T-cell therapy.

### Overview of Computational Pipelines for Automated Analysis of FC Data

The present disclosure generally concerns an approach to improving the automatic identification of hematological diseases using models that are trained to rapidly (i) distinguish between different cell types in a sample and then (ii) determine an appropriate prediction based on the distribution of immunophenotype collections across the sample. As further discussed below, the approach can be implemented via a framework that supports multiple computational pipelines—namely, a first computational pipeline for training a model to classify cells by distribution of immunophenotype collections and then classify a sample based on cell type distribution and a second computational pipeline for applying a trained model to FC data to produce an output indicative of a proposed diagnosis for a hematological disease.

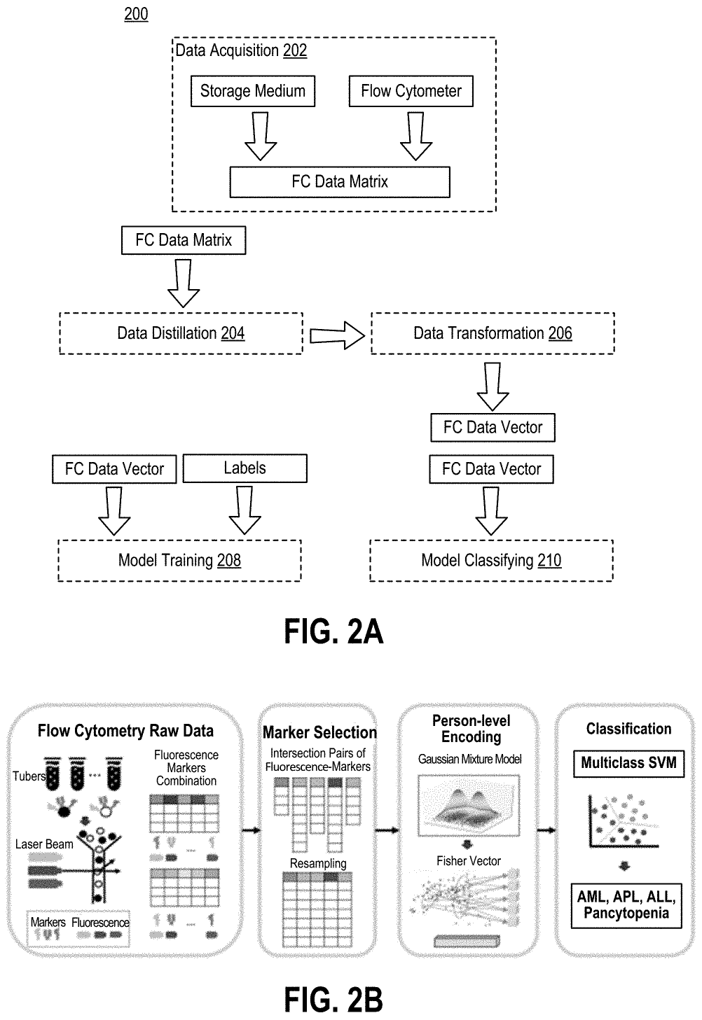

FIG. 2A includes a high-level illustration of a framework 200 that can be implemented by an analysis platform to acquire, process, and transform FC data to facilitate automated detection of hematological abnormalities that are indicative of hematological diseases. As further discussed below, the FC data can then be provided, as input, to a classification model for training purposes, or the FC data can then be provided, as input, to a classification model for classifying purposes. The classification model (also referred to as a “classifier model” or simply “classifier”) may be able to perform multiclass classification. Accordingly, when applied to FC data, the classification model may be able to produce multiple outputs that are representative of proposed diagnoses for different hematological diseases. As an example, an analysis platform may utilize a classification model for a multi-dimensional multicolor flow cytometry (MFC) phenotype that is trained using, for example, deep neural networks (DNNs) or support vector machines (SVMs) in combination with Gaussian mixture models (GMMs). In some embodiments, the classification model is a learned through supervised learning by analyzing an MFC dataset to develop an interpretation or understanding of MFC in order to objectively detect MRD. Supervised learning refers to a branch of AI in which datasets and accompanying labels are used to train models to reliably make predictions

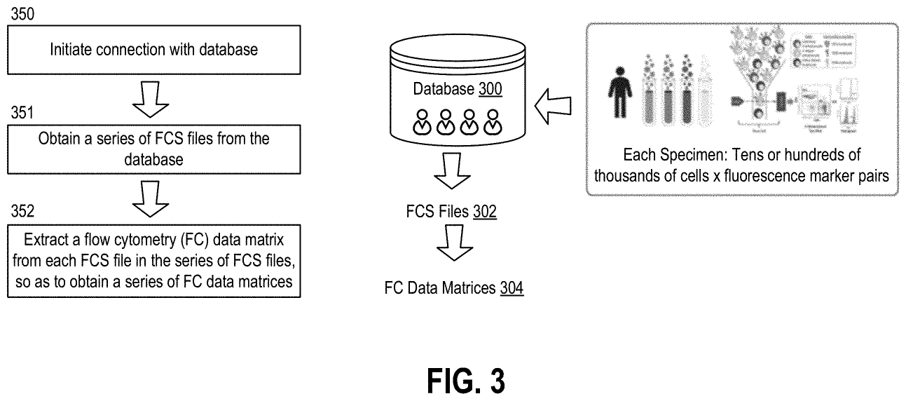

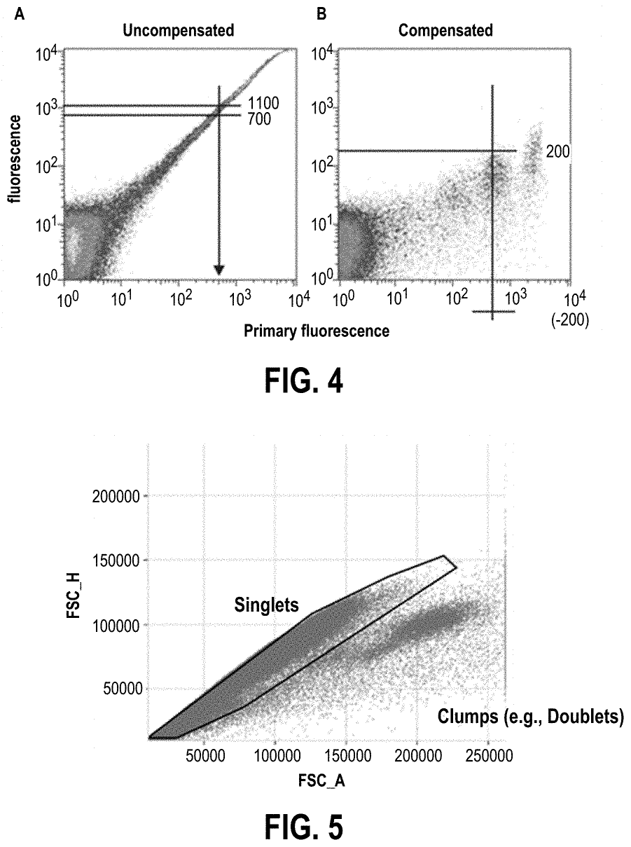

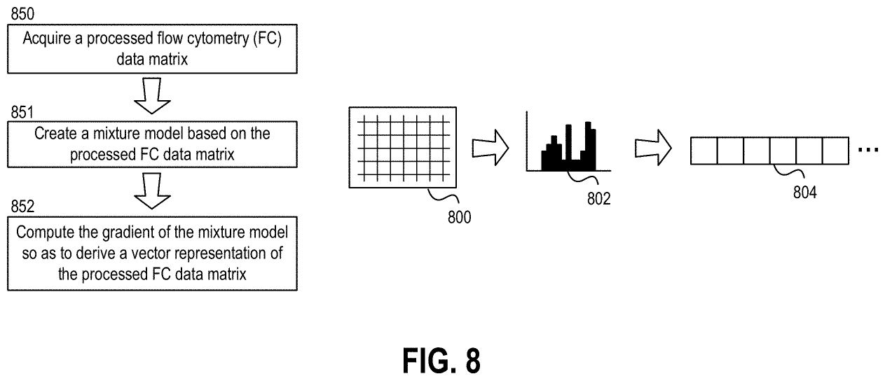

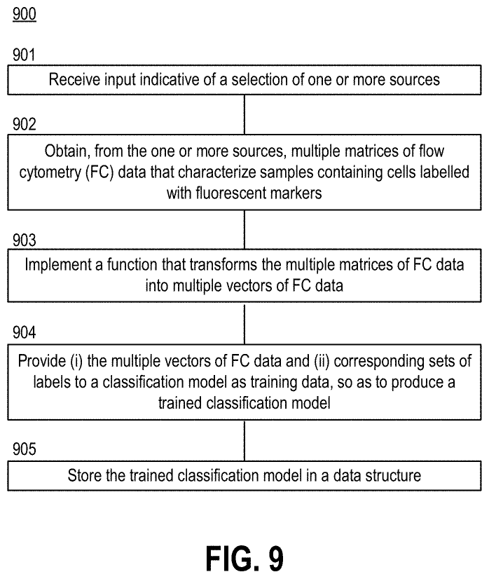

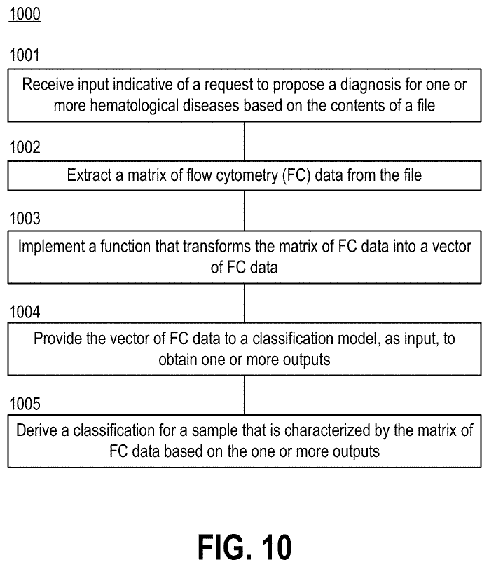

As shown in FIG. 2A, the framework 200 can include various stages. These stages may include a data acquisition stage 202, a data distillation stage 204, and a data transformation stage 206. The data acquisition stage 202 is further discussed below with reference to FIG. 3, the data distillation stage 204 is further discussed below with reference to FIGS. 4-7, and the data transformation stage 206 is further discussed below with reference to FIG. 8. Upon completing the data transformation stage 206, the analysis platform may provide the output to a classification model for training purposes 208 or classifying purposes 210. Training and classifying are further discussed below with reference to FIGS. 9 and 10, respectively.

In the data acquisition stage 202, an analysis platform may obtain FC data that characterizes a sample containing cells labelled with fluorescent markers from a source. The FC data may be included in a file that is formatted in accordance with the Flow Cytometry Standard (FCS). FCS is a file format standard for the reading and writing of data from FC experiments. The file format describes a file that is a combination of textual data that is followed by binary data, and the order of the file format is normally as follows: (1) header segment, (2) text segment, (3) data segment, (4) optional analysis segment, (5) cyclic redundancy check (CRC) value, and (6) optional other segments. The FC data may be representative of a matrix of measurements over M wavelengths by N parameters, where M and N are integer values, that can be extracted from the data segment of the FCS file. The parameters may include light scatter parameters and/or fluorescent marker parameters.

In some embodiments, the source from which the FC data is obtained by the analysis platform is the flow cytometer that generates the FC data. In other embodiments, the source is a storage medium that is accessible to the analysis platform, for example, via a network. The storage medium may be associated with an entity that manages the flow cytometer or another entity. In some embodiments, the storage medium is publicly accessible (e.g., via the Internet). In such embodiments, to obtain the FC data from the storage medium, the analysis platform may initiate a connection with the storage medium via a data interface (e.g., an application programming interface). In other embodiments, the storage medium is privately maintained and managed. For example, the storage medium may include proprietary clinical data that is generated by a healthcare system over time, and the analysis platform may be granted access to the storage medium in accordance with an agreement between the healthcare system and an entity that manages the analysis platform.

In the data distillation stage 204, the analysis platform can process the FC data in preparation for further handling. The nature of the data distillation stage 204 may depend on the form of the FC data obtained by the analysis platform. Assume, for example, that the analysis platform extracts an FC data matrix from an FCS file as discussed above. In such embodiments, the analysis platform can process the values included in the FC data matrix by performing a compensation operation, gating operation, and/or normalization operation as further discussed below. At a high level, the data distillation stage 204 may ensure that the analysis platform can analyze large batches of FC data in a consistent, accurate manner in relative short intervals of time. Because processing occurs before the analysis platform examines the FC data so as to gain insights therefrom, the data distillation stage 204 may also be preferred to as the “data preprocessing stage” or simply “data processing stage.”

In the data transformation stage 206, the analysis platform can transform the FC data into a form that is more suitable for further handling. For example, the analysis platform may implement a function that transforms the FC data matrix into a multi-dimensional vector using ML algorithms. Thus, the analysis platform may convert the FC data matrix into an FC data vector.

This FC data vector can be used in different ways depending on the computational pipeline that is presently being implemented or executed by the analysis platform.

Assume, for example, that the analysis platform is interested in training a classification model to identify hematological abnormalities that are indicative of a hematological disease. In such a scenario, the analysis platform may provide (i) the FC data vector and (ii) a set of labels that indicate, for each cell characterized in the vector, a pattern of immunophenotype collections to the classification model for training purposes 208. For example, the labels may indicate, for each cell characterized in the vector, a disease state, a disease status, or a physiological state (also referred to as a “pathological state”). Generally, the FC data vector is one of multiple FC data vectors that are provided to the classification model for training purposes 208, and the multiple FC data vectors may correspond to the different hematological diseases that the classification model is being trained to classify. Thus, the classification model may learn how to classify samples among a plurality of hematological diseases by learning, based on FC data vectors provided as input, the immunophenotypes that are representative of each of the plurality of hematological diseases.

Alternatively, the analysis platform may be interested in applying the classification model to the FC data vector for classification purposes 210. In such a scenario, the analysis platform may provide the FC data vector to the classification model as input, so as to obtain an output that is indicative of a proposed diagnosis for a hematological disease. As further discussed below, the classification model may be able to classify the FC data vector generated for a given sample (and thus a given patient) among more than one hematological disease in some embodiments. In such embodiments, the classification model may produce multiple outputs, each of which may be representative of a proposed diagnosis for a different hematological disease.

FIG. 2B illustrates how the framework shown in FIG. 2A can be used to (i) acquire “raw” FC data that is associated with a patient, (ii) select intersecting or interrelating parameters (e.g., fluorescent marker parameters), (iii) transform the “raw” FC data through patient-level encoding (e.g., using GMMs and Fisher Vectorization), and then either (iv) classify the patient by applying a classification model (e.g. a multiclass SVM) to the transformed FC data or (v) train a classification model (e.g., a multiclass SVM) to do the same. At a high level, FIG. 2B represents an overview of the framework that provides the general steps of the aforementioned computational pipelines. The nature of the training may depend on the targeted task, however. For example, step (ii) could be implemented via any of resampling, padding, or selecting fluorescent marker parameters (e.g., based on human knowledge or outputs produced by models) to derive the feature dimensions. If the targeted task involves patients with different panels of fluorescent markers, then step (ii) may be implemented to match the feature dimensions across the respective FC data from different panels. Additionally, step (ii) may involve an approach in the raw FC data is formed into a matrix that includes training data and testing data. Therefore, the encoding and classifying can be conducted with consideration of all of the fluorescent marker parameters simultaneously.

### A. Data Acquisition

FIG. 3 includes a high-level illustration of a process by which FC data is obtained from a source. Here, the source is a database 300 in which one or more flow cytometers are able to store the FC data that is generated through experimentation. This process may be performed by an analysis platform as part of a data acquisition step (e.g., data acquisition step 202 of FIG. 2A). At a high level, this is the process by which the analysis platform can acquire FC data that can be used to train a classification model to classify samples based on an analysis of cells that have been characterized by a flow cytometer. Similarly, this is the process by which the analysis platform can acquire FC data to which the classification model can be applied to generate one or more outputs (e.g., proposed diagnoses).

Generally, the database 300 has entries that include FC data for different specimens (and thus different patients) tested through experimentation. In FIG. 3, the entries include FCS files 302 in which FC data associated with the corresponding samples (and thus corresponding patients) are stored. Note, however, that FC data could be stored in the database 300 in another format.

The database 300 may be one of multiple databases from which the analysis platform is able to obtain FC data. Assume, for example, that the analysis platform is interested in acquiring FC data that is generated by flow cytometers located in different healthcare facilities associated with different healthcare systems. In such a scenario, the analysis platform may be permitted to access (i) a first database in which FC data generated by a first flow cytometer is stored and (ii) a second database in which FC data generated by a second flow cytometer is stored. Thus, the analysis platform may be able to obtain, sequentially or simultaneously, FC data from more than one source. Generally, the FC data stored in different databases will be associated with different sets of patients, though there may be some overlap (e.g., a patient could have one sample examined by a first flow cytometer associated with a first healthcare system and another sample examined by a second flow cytometer associated with a second healthcare system).

Generally, a flow cytometer will process a high number fluorescent markers simultaneously, in addition to several forward and side scattering properties. For example, the FC data generated during an experiment may include 17-23 channels, where 6 channels correspond to the forward and side scattering properties while the remaining channels correspond to different fluorescent marker properties. The forward and side scattering properties (also referred to as “optical properties” or “optical parameters”) may include forward scatter area (FSC-A), forward scatter width (FSC-W), forward scatter height (FSC-H), side scatter area (SSC-A), side scatter width (SSC-W), and side scatter width (SSC-H). Meanwhile, the fluorescent marker properties (also referred to as “fluorescent marker parameters”) may include CD117_PerCP-Cy5-5-A, KAPPA_FITC-A, HLA-DR_V450-A, CD38_APC-H7-A, and CD12_PE-A, among others. Accordingly, a single experiment can yield a large dataset.

When a flow cytometer analyzes a sample, FC data will be generated as an output. The FC data may be in the form of a matrix that has more than one dimension. For example, the FC data may comprise FSC signals (e.g., FSC-A, FSC-W, or FSC-H signals), SSC signals (e.g., SSC-A, SSC-W, or SSC-H signals), or fluorescence signals, and each of these signals may be treated as a separate dimension. Characteristics of these signals may also be treated as dimensions. Examples of characteristics include amplitude, frequency, amplitude variations, frequency variations, time dependency, space dependency, and the like. Moreover, the fluorescence signals may include red fluorescence signals, green fluorescence signals, or fluorescence signals in one or more other colors. Generally, the matrix will have at least three dimensions (and could have seven or more dimensions). For standardization purposes, the FC data may be presented in two-dimensional matrix form with individual signal values for training, validating, or testing in columns and features presented in rows. This FC data matrix may be exported from the flow cytometer in an FCS file.

As mentioned above, the analysis platform may apply a classification model to FC data that is extracted from an FCS file in order to classify individual cells and then classify the sample as a whole (e.g., as being representative of a hematological disease). In its raw form, however, the FC data can be difficult for the classification model to handle. For example, significant computational resources may be necessary for the classification model to expeditiously handle the FC data when in matrix form. Accordingly, the classification model may instead be trained to operate on FC data that has been transformed or converted into another form that can be more readily handled by the classification model.

While the process by which an FC data matrix can be transformed is discussed below with reference to FIG. 8, the analysis platform may extract the FC data matrix upon obtaining the FCS file as shown in FIG. 3. Accordingly, the analysis platform may initiate a connection with a database (step 350) to which one or more flow cytometers are able to upload FCS files, obtain a series of FCS files 302 from the database 300 (step 351), and then extract an FC data matrix from each FCS file (step 352), so as to obtain a series of FC data matrices 304. In embodiments where the analysis platform is interested in classifying a sample rather than training the classification model, the analysis platform may only obtain a single FCS file from the database.

### B. Data Distillation

Flow cytometers measure cell type based on the fluorescence response of an antibody expression as discussed above. Depending on the intended application, the number of cells for which fluorescence is measured during an experiment can range from several thousand to several million. For this reason, FC datasets (e.g., in the form of matrices) that are generated by flow cytometers can be very large.

Conventionally, these large FC datasets have been dealt with using dimensional reduction techniques that result in the production of scatter plots of individual values (e.g., for FSC, SSC, and fluorescence). While some information regarding clustering may be shown on these scatter plots, healthcare professionals are normally responsible for defining the “gates” that identify different regions on these scatter plots.

Rather than suggest clusters of cells on the cell level, the analysis platform may instead encode the large volumes of cell-level data as a patient-level representation to be used for automatic classification. To accomplish this, the analysis platform may employ an approach to encoding FC data that relies on ML-based techniques, such as GMMs and Fisher Vectorization, to aggregate the FC data for different levels of recognition tasks. The training of GMM models involves concatenating all cell-level data from all patients represented in the FC data used for training. Therefore, the approach can consume significant computational resources. Accordingly, downsampling and/or pooling may be employed in order to save on computational resources. The downsampling can be implemented by selecting a subset of data (e.g., by uniformly sampling the data), while pooling can be implemented by statistically representing sets of cells that are aggregated together based on the assumption that the processed data is still likely to form a similar distribution as the original data. For example, the analysis platform may represent sets of cells (e.g., of 3, 5, or 10 cells) with a mean vector in order to reduce memory consumption. An important aspect of recognition performance is that the FC data provided to the classification model as input is high quality. For this reason, the analysis platform may distill or process FC data before the FC data is further handled (e.g., transformed from matrix form to vector form).

As part of the data distillation stage, the analysis platform may perform (i) a compensation operation, (ii) a gating operation, and (iii) a normalization operation. Each of these operations is further discussed below.

Compensation is the process by which the analysis platform attempts to obtain the pure signal of each fluorescence intensity by eliminating the spillover signal from other fluorescence intensities included in an FC dataset. Thus, compensation is meant to ensure that laser performance of the flow cytometer is within an appropriate range. FIG. 4 illustrates how the spillover signal from other fluorescence intensities can bias the pure signal of the primary fluorescence intensity that is presently of interest. This can (and often does) lead to improper results when an individual is manual gating the fluorescence intensities populated on a scatter plot. To address this issue, individuals have historically run compensation beads through the flow cytometer before any experiments are performed in order to establish a compensation setting. Said another way, compensation beads can be run through the flow cytometer without any sample in an attempt to establish the spillover signal. That way, the flow cytometer will generate a spillover matrix that can later be used to calculate, infer, or otherwise determine the pure signal of each fluorescence intensity. The spillover matrix is generally saved by the flow cytometer in the text segment of each FCS file that is generated over an interval of time.

When the analysis platform obtains an FCS file, the analysis platform may not only extract the FC dataset from the data segment but can also extract the spillover matrix from the text segment. The analysis platform can then use the spillover matrix to perform a compensation operation. Said another way, the analysis platform can utilize the spillover matrix to produce a compensated FC dataset from the raw FC dataset extracted from the FCS file. Generally, the spillover matrix is an n×n matrix where “n” is the number of fluorescent markers associated with the corresponding sample. Considering each row as the raw measurement of the corresponding fluorescent marker, then each number in the same row may be representative of the contribution of a fluorescent marker to the measurement. This contribution is referred to as the “spillover coefficient” with a maximum value of one. Therefore, the diagonal elements of spillover matrix are all one, while the remaining numbers are between zero and one. The spillover matrix can be used to calculate the compensated measurement of each fluorescent marker by multiplying the inversion of the spillover matrix with the uncompensated data matrix for each fluorescent marker.

Note that the compensation operation may not be performed in every instance. For example, compensation may only be necessary when the analysis platform determines that the quality of the FC dataset is insufficient for training or classifying. The analysis platform may establish the quality based on an analysis of the raw FC dataset. For instance, the analysis platform may attempt to determine whether density, spread, or absolute value of measurements included in the FC dataset satisfy criteria that collectively define quality. As an example, the analysis platform may determine through computational analysis that an FC dataset similar to the one plotted in the scatter plot in FIG. 4 that is labeled “Uncompensated” has sufficient quality, while the analysis platform may determine through computational analysis that an FC dataset similar to the one plotted in the scatter plot in FIG. 5 that is labeled “Compensated” has sufficient quality. Better quality may be desired so that the analysis platform can perform automated analysis with better accuracy.

Singlets gating (or simply “gating”) is the process by which inaccurate signals of non-specific binding events or doublets are removed from an FC dataset before its contents are actually gated. Assume, for example, that two cells are simultaneously measured by the flow cytometer because those cells are aligned while passing through the laser beam. To ensure that the corresponding measurement generated by the flow cytometer does not affect performance of the classification model, it may be desirable to remove the corresponding measurement from the FC dataset.

This process—also referred to as “doublet exclusion”—has historically involved plotting the height or width against the area for FSC or SSC. As an example, FIG. 5 illustrates how a scatter plot can be generated with FSC-H along the y-axis and FSC-A along the x-axis to facilitate manual singlets gating. To perform gating, individuals have traditionally identified the region of singlets by defining a region on the scatter plot. This approach relies on the linearity between FSC-H and FSC-A, so the region is commonly drawn along a straight line that is roughly equivalent to the diagonal line as shown in FIG. 5.

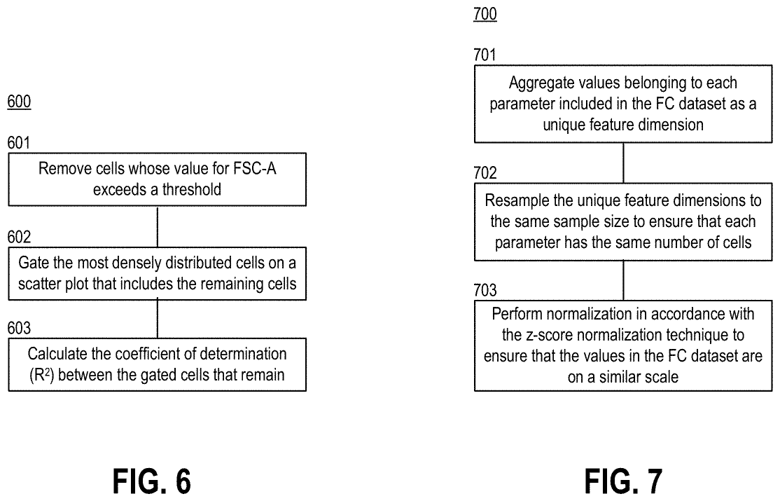

To eliminate the ambiguity inherent in manual gating, the analysis platform may implement a function that performs gating or doublet discrimination in an automated manner. This function may help ensure that each value in the FC dataset corresponds to a single cell. FIG. 6 includes a flow diagram of a process 600 for automatically performing singlet gating. Initially, the analysis platform may remove cells whose value for FSC-A reaches a threshold (step 601). As an example, the analysis platform may remove all cells whose value for FSC-A is the maximum value. The maximum value may be 2¹⁸, which is the highest value possible for FC data in linear scale. As another example, the analysis platform may remove all cells whose value for FSC-A is within the top two, three, or five percent of values across the FC dataset. Accordingly, the threshold may be programmed in instructions that are executable by the analysis platform, or the threshold may be dynamically determined by the analysis platform based on the FC dataset. FSC-H and FSC-A are displayed in linear scale when performing singlets gating, so this step may be performed by the analysis platform to emulate the actions of a healthcare professional. More specifically, this step may be automatically performed to remove the cells that unnaturally “stick” to the right side of the scatter plot as can be seen in FIG. 5, since those cells would not be included in the region if manually defined by the healthcare professional.

The analysis platform can then gate the most densely distributed cells on a scatter plot that includes the remaining cells (step 602). More specifically, the analysis platform can produce a scatter plot based on FSC-H and FSC-A values that are included in the compensated FC dataset for the remaining cells, and then the analysis platform can gate the most densely distributed cells on the scatter plot. For example, the analysis platform may gate the 90, 95, or 98 percent most densely distributed cells on the scatter plot. This percentage may be referred to as the “gating fraction.” Due to the high linearity between FSC-H and FSC-A, these gates should capture mostly singlets rather than doublets.

Thereafter, the analysis platform can calculate the coefficient of determination (R²) between the gated cells that still remain after step 602 (step 603). If the R² value exceeds an upper threshold (e.g., 0.80, 0.85, or 0.90), the function implemented by the analysis platform may return the data in the FC dataset that is associated with those cells and then terminate. Otherwise, the function may instruct the analysis platform to perform steps 602-603 repeatedly with the gating fraction decreasing by a predetermined amount (e.g., 2 percent, 3 percent, 5 percent, or 10 percent) each time until the R² value exceeds the upper threshold. If the R² value still does not exceed the upper threshold when the gating fraction hits a lower threshold (e.g., 70 percent, 75 percent, or 80 percent), the analysis platform can generate an alert that specifies the sample lacks linearity between FSC-H and FSC-A. Because this could lead to further issues with using the FC dataset (e.g., in training or classifying), the analysis platform may simply return the raw FC dataset or the compensated FC dataset.

AI-assisted analysis represents an attractive option for classifying samples represented by FC datasets in a more systematic and consistent manner than is possible when individuals are responsible for manually examining the FC datasets. However, it may be necessary for institutions to adhere to a protocol in order for the AI-assisted analysis to widely impact clinical and diagnostic practices.

Normalization is the process by which the analysis platform can overcome the issue of non-standardized handling of FC dataset. Normalization may be useful as a means of improving the performance and training stability of the classification model to which an FC dataset is provided as input, for either training or classifying purposes. FIG. 7 includes a flow diagram of a process 700 for normalizing an FC dataset that is extracted from an FCS file. As mentioned above, the analysis platform will normally perform the normalization operation after performing the compensation and gating operations to ensure that improper and inaccurate values are removed before those values are normalized.

As mentioned above, the FC dataset will normally include values for multiple parameters. For example, the FC dataset may include values for one or more light scatter parameters in addition to values for one or more fluorescent marker parameters. As part of the normalization operation, the analysis platform may initially aggregate the values belonging to each parameter as a unique feature dimension (step 701). The analysis platform can then resample the unique feature dimensions to the same sample size to ensure that each parameter has the same number of cells (step 702). Note that, in some embodiments, the analysis platform may resample the unique feature dimensions so that the parameters have roughly the same number of cells (e.g., within 2 percent, 5 percent, or 10 percent) rather than the exact same number of cells.

For example, values for a fluorescent marker parameter (e.g., CD56-APC) may be aggregated across multiple samples as a single parameter to ensure that the number of values (and thus number of cells) meets a count criterion determined through resampling. As another example, values for a light scatter parameter (e.g., FSC-A or SSC-A) may be aggregated across multiple samples and then downsampled to ensure that the number of values (and thus number of cells) meets a count criterion determined through resampling. At a high level, the count criterion may be representative of the number of samples determined to be appropriate by the analysis platform.

Through normalization, the analysis platform may be able to generate a processed FC dataset that can be used as input by other elements of the framework as further discussed below. For example, the analysis platform may perform normalization in accordance with the z-score normalization technique to ensure that the values in the FC dataset are on a similar scale (step 703), so as to produce a processed FC dataset. The z-score normalization technique is a variation of scaling that represents the number of standard deviations away from the mean. The formula for calculating the z-score of a value (x) is shown below:

x′=(x−μ)/σ, Eq. 1

where μ is the mean and σ is the standard deviation. The z-score normalization technique can be used to ensure that the distributions have a mean of zero and a standard deviation of one, and therefore is useful when there are a few outlier values but not so many that more drastic measures (e.g., clipping) are needed. Other normalization techniques could also be used by the analysis platform. For example, the analysis platform may implement scaling to a range, clipping, or log scaling instead of, or in addition to, the z-score.

Accordingly, to process an FC dataset extracted from an FCS file, the analysis platform may perform (i) a compensation operation, (ii) a gating operation, and (iii) a normalization operation. Thereafter, the FC dataset could be stored in a storage medium that is accessible to the analysis platform, or the FC dataset could be further handled by the analysis platform in accordance with the appropriate computational pipeline. Other steps could also be performed. For instance, the analysis platform may generate a visual indicium of values (e.g., FSC-H and FSC-A values) that remain in the FC dataset after processing as a means of allowing an individual to review how the analysis platform automatically compensated, gated, and normalized the FC dataset. As an example, the analysis platform may generate a report that includes analyses of the values that remain in the FC dataset after processing. As another example, the analysis platform may generate a scatter plot that includes the values that remain in the FC dataset after processing. Regardless of its form and contents, the visual indicium could be posted to an interface generated by the analysis platform for review by the individual.

Processing FC datasets in the prescribed manner ensures that the analysis platform can analyze large amounts of data with improved quality—and with the effects of signal drift largely, if not entirely, alleviated—in a relatively short period of time.

### C. Data Transformation

While useful insights can be gained through analysis of raw FC data or “processed” FC data, a classification model may struggle to handle these data, especially if the classification model is tasked with handling the data for tens or hundreds of specimens over a short period of time. Accordingly, the analysis platform may transform processed FC data into a form that is more suitable for further use. In particular, the analysis platform may transform processed FC data into a form that is well suited for input into the classification model.

FIG. 8 includes a high-level illustration of a process by which processed FC data is transformed from its matrix form into a vector. This process may be performed by an analysis platform as part of a data transformation step (e.g., data transformation step 206 of FIG. 2A). At a high level, the analysis platform may perform the process to convert processed FC data into a form that can be more easily handled by a classification model. As an example, the processed FC data may be transformed into a vector 804 using Fisher vector encoding and a GMM distribution. After transformation has occurred, the representation of each sample may be a high-dimensional vector that characterizes the corresponding patient's specimen phenotype. This representation can be readily used by different types of classification models, including SVMs, DNNs, and random forests.

Initially, the analysis platform can acquire a processed FC data matrix 800 (step 850). The processed FC data matrix 800 is normally produced by the analysis platform through processing of a “raw” FC data matrix as discussed above with reference to FIGS. 4-7. Accordingly, the processed FC data matrix 800 may be readily available to the analysis platform, and the process may simply be the next stage in a framework (e.g., framework 200 of FIG. 2A) that is implemented by the analysis platform.

Alternatively, the analysis platform could acquire the processed FC data matrix 800 from elsewhere. For example, the analysis platform may obtain FCS files generated by flow cytometer(s) on a continual or periodic basis. As discussed above, the analysis platform may process the “raw” FC data matrix that included in each FCS file. However, rather than immediately transform the processed FC data matrix, the analysis platform may store the processed FC data matrix in a storage medium for future use. Thus, the analysis platform may not immediately perform the process shown in FIG. 8 after processing a “raw” FC data matrix. Instead, the analysis platform may store the processed FC data matrix so that it can implement a “batch training” scheme where training occurs periodically (and processed FC data matrices only need to be transformed periodically).

The analysis platform can then create a mixture model 802 based on the processed FC data matrix 800 (step 851). At a high level, a mixture model is a probabilistic model that is intended to represent the presence of cell types within the processed FC data matrix by clustering comparable values. Thus, the mixture model 802 may correspond to the mixture distribution that represents the probability distribution of cell type observations across the entire sample represented by the processed FC data matrix 800. One example of a mixture model is a GMM,

Thereafter, the gradient of the mixture model 802 can be computed using an ML algorithm to derive a vector representation 804 for the processed FC data matrix 800 (step 852). This gradient-based feature space transformation may rely on a distance function to estimate the relationship between the cell in the processed FC data matrix 800 and the clusters defined by the GMM. Fisher kernel distance that is used in Fisher Vectorization is one example of a distance function that measures higher-order relationships based on the probabilistic cluster distribution. Therefore, the derived vector representation can characterize the complex cell distribution of the processed FC data matrix using the relationship to each cluster. For example, the analysis platform may compute a Fisher vector using the mixture model to construct the vector 804. While the mixture model 802 may attempt to cluster comparable values, Fisher Vectorization—when implemented by the analysis platform—may further encode the processed FC data matrix based on the trained parameters of the mixture model 802.

The dimensions of the vector 804 may be based on the dimensions of the processed FC data matrix 800 and the cluster number (also referred to as the “mixture number”). Accordingly, if the processed FC data matrix 800 includes various dimensions as discussed above, then the vector 804 may be a high-dimensional vector. Each cell characterized in the processed FC data matrix 800 may be associated with multiple entries in the high-dimensional vector, and each of these entries may correspond to a different parameter (e.g., FSC, SSC, fluorescence intensity, and characteristics such as amplitude, frequency, and the like) to describe the relationship to the distribution of clusters in the GMM. The benefit of representing the underlying FC data as a high-dimensional vector follows partly from high dimensionality (e.g., n=17, 23, or more parameters multiplied by the mixture number, which can result in hundreds or thousands of dimensions) and partly from the ability to gain greater insight into the interconnections between different dimensions through learning.

With a GMM that is trained using an entire training dataset (e.g., comprised of multiple FC datasets), the analysis platform can compute the posterior probability of each cell-level FC dataset to determine the likelihood that the cell belongs to each “cluster” or “mixture” defined by the GMM. Fisher Vectorization can be used to transform the cell vectors by considering the posterior probability of each cluster along with the distance between the cell vector and a center vector created for each cluster. This distance used in Fisher Vectorization considers mean vectors, covariance matrices, and weights of the GMM, and therefore can represent the complex high-order relationship between the cell vector and each cluster. Fisher Vectorization is one example of an approach that weighs the distances via posterior probabilities. With the GMM parameters, other distance functions could also be applied to estimate the cell-to-cluster relationship. Finally, each FC dataset can be represented by an averaged cell representation that embeds the information about its posterior probabilities and its relationship to the clusters.

### D. Training

FIG. 9 includes a flow diagram of a process 900 for training a model to classify hematological diseases. Initially, an analysis platform can receive input indicative of a selection of one or more sources from which to obtain FC data (step 901). For example, the input may specify multiple databases in which separate sets of FCS files (e.g., associated with different patients, generated by different flow cytometers) are stored. As another example, the input may specify multiple flow cytometers from which FCS files are to be acquired. In embodiments where more than one source is selected, the FC data obtained from each source is normally related to different sets of patients. Patients could be included in both sets, however. Alternatively, the input may specify a single database or flow cytometer from which FCS files are to be acquired.

The analysis platform can then obtain, from the one or more sources, multiple matrices of FC data that characterize samples containing cells labelled with fluorescent markers (step 902). For example, the analysis platform may acquire multiple FCS files that are generated by flow cytometer(s) as mentioned above, and then the analysis platform may extract a matrix of FC data from each FCS file.

The nature of the multiple matrices of FC data may depend on the goal of the analysis platform in training the classification model. Assume, for example, that the analysis platform is interested in training the classification model to distinguish between four different hematological diseases. In such a scenario, the samples that correspond to the multiple matrices of FC data may be known to correspond to confirmed instances of those four different hematological diseases. Accordingly, the analysis platform may acquire at least one matrix of FC data for each hematological disease of interest.

While the contents of each matrix of FC data may vary, the structure tends to be fairly consistent. For example, each matrix may include FSC values, SSC values, or fluorescence values over M wavelengths by N parameters, where M and N are integer values. Thus, each matrix could include a first set of FSC values, a second set of SSC values, or a third set of fluorescence values.

The analysis platform can then implement a function that transforms the multiple matrices of FC data into multiple vectors of FC data (step 903). When implemented, the function may independently transform each matrix of FC data into a corresponding vector of FC data. Generally, this is accomplished through the use of an ML algorithm. As an example, each matrix of FC data may be transformed into the corresponding vector of FC data using Fisher vector encoding and a GMM distribution as discussed above. In embodiments where the function converts the matrices of FC data through Fisher vector encoding, each vector may be the Fisher vector representation of the FC data included in the corresponding matrix.

Thereafter, the analysis platform can provide (i) the multiple vectors of FC data and (ii) corresponding sets of labels to the classification model as training data, so as to produce a trained classification model (step 904). Each set of labels may indicate a type of immunophenotype collection encoded or characterized in the corresponding vector, as well as a type of hematological disease of which the corresponding sample is representative. For example, each set of labels may indicate, for each cell characterized in the corresponding vector, a disease type, disease status, or physiological status. Accordingly, the labels may not help the classification model learn how to classify individual cells, but also how to classify an entire sample (e.g., among multiple hematological diseases) based on its distribution of immunophenotype collections. As mentioned above, if the vectors comprise FC data related to more than one hematological disease, then the classification model may be trained to distinguish between multiple hematological diseases (e.g., ALL, AML, APM, and pancytopenia).

In some embodiments, the multiple vectors of FC data and corresponding sets of labels are included in a larger training dataset that is used to train the classification model. This larger training dataset may further include information regarding one or more optical parameters and/or one or more fluorescent marker parameters. Examples of optical parameters include forward scatter area (FSC-A), forward scatter width (FSC-W), forward scatter height (FSC-H), side scatter area (SSC-A), side scatter width (SSC-W), and side scatter width (SSC-H). Meanwhile, examples of fluorescent marker parameters include CD117_PerCP-Cy5-5-A, KAPPA_FITC-A, HLA-DR_V450-A, CD38_APC-H7-A, and CD123_PE-A.

The analysis platform can then store the trained classification model in a data structure (step 905). As further discussed below, the analysis platform may subsequently use the trained classification model to produce classifications that are indicative of proposed diagnoses for different hematological diseases. As such, the analysis platform may programmatically associate the trained classification model with each hematological disease for which it can produce a proposed diagnosis. For example, the analysis platform may populate the data structure with identifiers (e.g., alphanumeric identifiers) that identify the hematological diseases for which the classification model is able to produce proposed diagnoses.

In sum, the multiple vectors of FC data and corresponding sets of labels indicating the type of immunophenotype characterized in the FC data may be fed into a classification model for training purposes. Thus, the multiple vectors may be representative of training data that can be used to train the classification model to classify a given sample among different hematological diseases. The training data used to train the classification model may include an assembly of high-dimensional vectors that are associated with different samples (and thus different patients). Once trained, the classification model may be able to classify a sample based on an analysis of its corresponding FC data to identify different patterns of immunophenotype collections and then determine whether the sample is representative of a hematological disease based on the sample-wide distribution of immunophenotypes.

### E. Classifying

FIG. 10 includes a flow diagram of a process 1000 for classifying a sample through the application of a classification model. Assume, for example, that an analysis platform receives input indicative of a request to propose a diagnosis for one or more hematological diseases based on the contents of a file (step 1001). This input may be representative of a selection of the file (or a corresponding patient) through an interface generated by the analysis platform, or this input may be representative of a receipt of the file (e.g., from a flow cytometer). The file may be formatted in accordance with FCS, as an example. In this situation, the analysis platform can extract FC data from the file in a first form and then transform the FC data into a second form that can be more easily handled by the classification model. For example, the analysis platform may extract a matrix of FC data from the file (step 1002). Then, the analysis platform can implement a function that transforms the matrix of FC data into a vector of FC data (step 1003). This function may be the same function discussed above with reference to step 903 of FIG. 9.

The analysis platform can then provide the vector of FC data to a classification model, as input, to obtain one or more outputs (step 1004). Each output may be representative of a proposed diagnosis for a different hematological disease. Thus, the analysis platform may be able to derive a classification for the sample that is characterized by the FC data based on the output(s) (step 1005). As mentioned above, the number of outputs that are produced by the classification model may be based on the number of hematological diseases for which training data was providing during a training phase. Normally, the classification model is trained to produce outputs for multiple hematological diseases upon being applied to the vector of FC data; however, the classification model could be trained to produce a single output for a hematological disease upon being applied to the vector of FC data. In embodiments where the classification model is associated with a single hematological disease, the analysis platform may apply multiple classification models that have been trained to classify different hematological diseases in accordance with the approach described herein. Additionally or alternatively, the number of outputs that are produced by the classification model may be based on the number of disease states defined for a given hematological disease and/or the number of numerical ranges defined for MRD.

Note that while the sequences of the steps in the processes described herein are exemplary, the steps can be performed in various sequences and combinations. For example, steps could be added to, or removed from, these processes. Similarly, steps could be replaced or reordered. Thus, the descriptions of these processes are intended to be open ended.

Additional steps may also be included in some embodiments. For example, the analysis platform may be able to derive a classification (e.g., a proposed diagnosis for a hematological disease) based on an output produced by a classification model as discussed above. In such a scenario, the analysis platform may be able to cause display of the classification on an interface that is accessible to a patient associated with the underlying FC data. Similarly, the analysis platform may be able to cause display of the classification on an interface that is accessible to a healthcare professional. In some embodiments, the analysis platform is able to interface with the central computing system of a healthcare provider. For example, the analysis platform may be able to access the central computing system via a data interface to access FC data. In such a scenario, the analysis platform may be able to automatically populate the classification into the electronic health record (EHR) of the corresponding patient. For example, the analysis platform may transmit the classification to the central computing system with an instruction to populate the classification into the EHR for recordation purposes.

### E. Use Case

In a typical setting, the approach described herein may be used to further examine FC data of interest. As an example, the FC data of interest may correspond to a suspicious laboratory result for which a healthcare professional would like further information before determining an appropriate course of action. To accomplish this, the analysis platform may apply a classification model to the FC data of interest. Assume, for example, that the classification model is trained to classify different patterns of immunophenotype collections so as to distinguish between multiple hematological diseases (e.g., ALL, AML, APM, and pancytopenia). With fast and accurate classification by the classification model, a healthcare professional may be able to select an appropriate treatment.

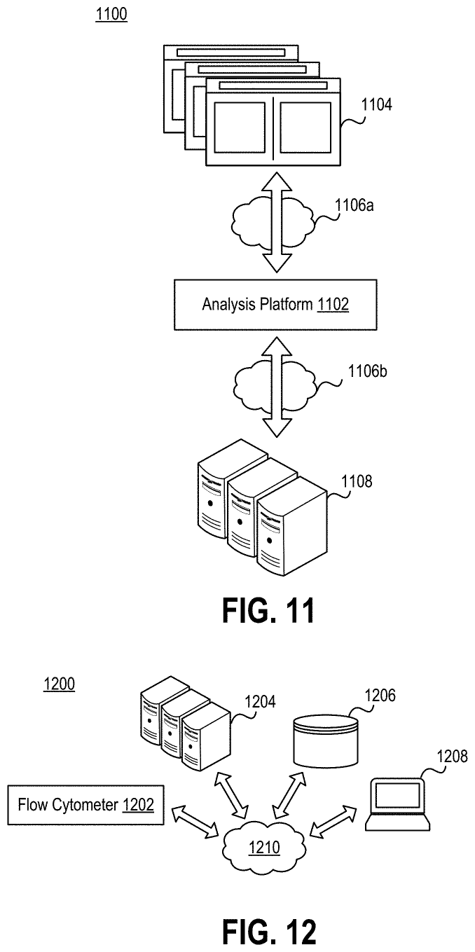

As discussed above, the classification model may be implemented by the analysis platform so as to classify a disease or a physiological status by type in an automated manner. Generally, the analysis platform is part of an automatic classification system (or simply “system”) as further discussed below with reference to FIGS. 11-12. The system may comprise a flow cytometer, a network-accessible server system, a datastore, and a computing device (also referred to as an “electronic device” or “user device”). In some embodiments, the entire system is implemented within a single housing.

Generally, the process by which a sample is automatically classified by the analysis platform begins with an individual preparing samples for insertion into a flow cytometer. For example, the individual may prepare a series of tubes, each of which includes a different sample. Each tube may be subject to a panel of different suitable fluorescent markers. As the series of tubes are examined by the flow cytometer, FC data is generated that is encoded into separate files. As discussed above, these files can be used by the analysis platform to train a classification model to produce outputs that are diagnostically useful.

To ensure good coverage, the training dataset that is used by the analysis platform to train the classification model may be based on, or derived from, a large number of files. For example, the training dataset may include FC data for several thousand (e.g., 1,000, 2,000, or 4,000) patients that are known to have been diagnosed with ALL, AML, or APL. Each sample may be associated with a single patient, though a single sample could be associated with multiple tubes (and thus multiple files generated by the flow cytometer). For example, a sample set of roughly 1,000-2,000 samples may be associated with roughly 4,000-12,000 tubes due to size constraints.

To illustrate its usefulness, the framework described herein was used to develop a four-category classification model using FCS files generated by a flow cytometer, namely, FASCantoll from Becton Dickinson Bioscience. The FCS files corresponded to roughly 550 bone marrow samples with about 100 cases of ALL, about 200 cases of AML, and about 200 cases of pancytopenia without hematological disease. These diagnoses were based on routine morphology, cytogenetic, molecular, and clinical findings. GMMs were built using the raw fluorescence intensities for the antibody-fluorochrome conjugates employed in ≥90 percent of samples for each of the four categories and light scatter parameters. For each GMM, the gradient of each light scatter parameter was computed using Fisher vectorization to derive a high-dimensional representation that was used to train the four-category classification model.

To evaluate performance, accuracy (ACC) was used and defined as the concordance rate between the diagnoses made through the manual and automated approaches. Furthermore, sensitivity and specificity were assessed based on the area under the receiver operating characteristic (ROC) curve, also referred to as the “AUC.”

Single-parameter analysis was performed first and found that FSC-A provided the highest accuracy in comparison to 36 other parameters, including 31 markers that are often used to measure performance in FC analysis. The complete list of parameters used in the study included FSC-A, SSC-H, CD117_PerCP-Cy5-5-A, FSC-H, KAPPA_FITC-A, HLA-DR_V450-A, CD38_APC-H7-A, CD123_PE-A, FSC-W, CD34_APC-A, CD19_PE-Cy7-A, CD2_V450-A, CD14_APC-H7-A, SSC-W, CD4_PerCP-Cy5-5-A, CD45_V500-A, CD64_PerCP-Cy5-5-A, CD7_FITC-A, CD8_APC-H7-A, CD10_APC-A, SSC-A, CD7_PE-A, CD19_PerCP-Cy5-5-A, and CD33_PE-Cy7-A. Accordingly, the parameters included optical parameters and fluorescent marker parameters.

Because an optical parameter (i.e., FSC-A) exhibited the best performance, all six optical parameters were studied and compared for the additive effect in terms of accuracy and AUC. The results are shown below in Table I. As can be seen in Table I, the combination three optical parameters (i.e., FSC-A, SSC-H, and SSC-W) exhibited a reasonable accuracy of 0.921 while the combination of all six optical parameters exhibited accuracy of 0.938. Analysis revealed that when an additional optical parameter (i.e., FSC-W), was included, the accuracy rose to 0.928 with an AUC of 0.990. Meanwhile, the addition of two optical parameters (i.e., FSC-W and FSC-H) only increased the accuracy to 0.940 with an AUC of 0.991. Accordingly, a classification model trained with as few as three parameters performed nearly as well as a classification model trained with all six parameters.

TABLE I
Accuracy and AUC values for different
combinations of optical parameters.

| Marker Combination | ACC | AUC |
|---|---|---|
| FSC-A, SSC-H, SSC-W | 0.921 | 0.985 |
| FSC-A, SSC-H, SSC-A | 0.911 | 0.979 |
| FSC-A, SSC-H, FSC-W | 0.910 | 0.984 |
| FSC-A, SSC-H, FSC-H | 0.906 | 0.981 |
| FSC-A, SSC-H, SSC-W, FSC-H | 0.925 | 0.989 |
| FSC-A, SSC-H, SSC-W, FSC-W | 0.928 | 0.990 |
| FSC-A, SSC-H, SSC-W, SSC-A | 0.925 | 0.987 |
| FSC-A, SSC-H, SSC-W, FSC-W, FSC-H | 0.940 | 0.991 |
| FSC-A, SSC-H, SSC-W, FSC-W, SSC-A | 0.940 | 0.990 |
| FSC-A, SSC-H, SSC-W, FSC-W, FSC-H, SSC-A | 0.938 | 0.991 |

Further investigation revealed that accuracy and AUC can be improved with selected fluorescent marker parameters without using all 37 fluorescent marker parameters that were tested. The results are shown below in Table II. As can be seen in Table II, the inclusion of a CD117 marker (i.e., CD117_PerCP-Cy5-5-A) resulted in an accuracy of 0.932 with an AUC of 0.983—a significant improvement over the two-parameters combination of FSC-A and SSC-H. The inclusion of another fluorescent marker parameter—namely, KAPPA_FITC-A—resulted in the accuracy increasing to 0.948 with an AUC of 0.990. The inclusion of HLA-DR_V450-A, CD38_APC-H7-A, and CD123_PE-A also provided better accuracy and AUC as can be seen in Table II.

TABLE II
Accuracy and AUC values for different combinations of optical
parameters and fluorescent marker parameters.

| Parameter | | | |
|---|---|---|---|
| Count | Marker Combination | ACC | AUC |
| 1 | FSC-A | 0.770 | 0.900 |
| 2 | FSC-A, SSC-H | 0.885 | 0.967 |
| 3 | FSC-A, SSC-H, CD117_PerCP-Cy5-5-A | 0.932 | 0.983 |
| 5 | FSC-A, SSC-H, CD117_PerCP-Cy5-5-A, | 0.949 | 0.990 |
| | FSC-H, KAPPA_FITC-A | | |
| 7 | FSC-A, SSC-H, CD117_PerCP-Cy5-5-A, | 0.953 | 0.992 |
| | FSC-H, KAPPA_FITC-A, HLA-DR_V450- | | |
| | A, CD38_APC-H7-A | | |
| 8 | FSC-A, SSC-H, CD117_PerCP-Cy5-5-A, | 0.957 | 0.992 |
| | FSC-H, KAPPA_FITC-A, HLA-DR_V450- | | |
| | A, CD38_APC-H7-A, CD123_PE-A | | |
| 4 | FSC-A, SSC-H, CD117_PerCP-Cy5-5-A, | 0.945 | 0.992 |
| | FSC-H | | |

### Overview of Analysis Platform

FIG. 11 illustrates a network environment 1100 that includes an analysis platform 1102. Individuals (also referred to as “users”) can interface with the analysis platform 1102 via interfaces 1104. For example, a user may be able to access an interface through which information regarding a patient, as well as a proposed diagnosis for the patient, can be viewed. These interfaces 1104 may permit users to interact with the analysis platform 1102 as it implements the framework described herein. The term “user,” as used herein, may refer to a person who is interested in examining a proposed diagnosis, such as a patient or healthcare professional, or a person who is interested in developing, training, or implementing models.

As shown in FIG. 11, the analysis platform 1102 may reside in a network environment 1100. Thus, the computing device on which the analysis platform 1102 is implemented may be connected to one or more networks 1106a-b. These networks 1106a-b may be personal area networks (PANs), local area networks (LANs), wide area networks (WANs), metropolitan area networks (MANs), cellular networks, or the Internet. For example, the analysis platform 1102 may be indirectly connected to one or more flow cytometers via the Internet (e.g., via corresponding application programming interfaces), or the analysis platform 1102 may be directly connected to one or more flow cytometers (e.g., via corresponding tunnels). As another example, the analysis platform 1102 may be connected, either directly or indirectly, to storage mediums that are managed by respective healthcare systems. These storage mediums may be part of laboratory information systems, electronic health record systems, etc. Additionally or alternatively, the analysis platform 1102 can be communicatively coupled to one or more computing devices over a short-range wireless connectivity technology, such as Bluetooth®, Near Field Communication (NFC), Wi-Fi® Direct (also referred to as “Wi-Fi P2P”), and the like.

The interfaces 1104 may be accessible via a web browser, desktop application, mobile application, or over-the-top (OTT) application. For example, a healthcare professional may be able to access an interface through which information regarding a patient can be input. Such information can include name, date of birth, symptoms, medications, and experiment results (e.g., in the form of an FCS file). With this information, the healthcare professional may be able to implement the framework to produce a classification that is representative of a proposed diagnosis. As another example, an individual may access an interface through which she can identify datasets and then monitor as the analysis platform 1102 implements the framework to train a classification model using the datasets. Accordingly, the interfaces 1104 may be viewed on computing devices such as mobile workstations (also referred to as “medical carts”), personal computers, tablet computers, mobile phones, wearable electronic devices, and the like.

In some embodiments, at least some components of the analysis platform 1102 are hosted locally. That is, part of the analysis platform 1102 may reside on the computing device that is used to access the interfaces 1104. For example, the analysis platform 1102 may be embodied as a desktop application that is executable by a mobile workstation accessible to one or more healthcare professionals. Note, however, that the desktop application may be communicatively connected to a server system 1108 on which other components of the analysis platform 1102 are hosted.

In other embodiments, the analysis platform 1102 is executed entirely by a cloud computing service operated by, for example, Amazon Web Services®, Google Cloud Platform™, or Microsoft Azure®. In such embodiments, the analysis platform 1102 may reside on a server system 1108 that is comprised of one or more computer servers. These computer servers can include models, algorithms (e.g., for processing FC data, generating reports, etc.), patient information (e.g., profiles, credentials, and health-related information such as age, date of birth, disease classification, healthcare provider, etc.), and other assets. Those skilled in the art will recognize that this information could also be distributed amongst the server system 1108 and one or more computing devices. For example, some data that is generated by the computing device on which the analysis platform 1102 resides may be stored on, and processed by, that computing device for security or privacy purposes.

FIG. 12 includes a diagram illustrating one example of a system 1200 that is able to automatically classify different patterns of immunophenotype collections so as to identify hematological diseases. The system 1200 may comprise a flow cytometer 1202 that is communicatively connected to an analysis platform. Here, the analysis platform is implemented on a network-accessible server system 1204, though the analysis platform could be implemented elsewhere as mentioned above. The system 1200 also comprises a datastore 1206 and a computing device 1208. The computing device 1208 may be one of multiple computing devices that can be used to interface with the analysis platform. For example, in embodiments where multiple individuals (e.g., the healthcare professionals employed by a healthcare system) are able to interface with the analysis platform, more than one computing device may be part of the system 1200. As shown in FIG. 12, the components of the system 1200 may be communicatively connected to one another, either directly or indirectly, via a network 1210. Additionally or alternatively, the components of the system 1200 may be communicatively connected to one another via physical communication interfaces.

As mentioned above, the functionality of the network-accessible server system 1204, datastore 1206, and computing device 1208 could be implemented in a single device. Similarly, the functionality of the flow cytometer 1202, network-accessible server system 1204, database 1206, and computing device 1208 could be implemented in a single flow cytometer, in which case the flow cytometer may be referred to as a “combined flow cytometer” or “comprehensive flow cytometer.”

### Processing System

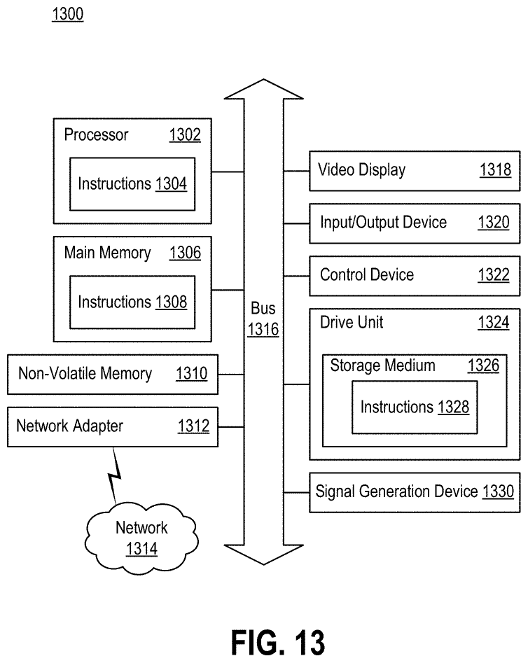

FIG. 13 is a block diagram illustrating an example of a processing system 1300 in which at least some operations described herein can be implemented. For example, components of the processing system 1300 may be hosted on a computing device that includes an analysis platform (e.g., analysis platform 1102 of FIG. 11). As another example, components of the processing system 1300 may be hosted on a flow cytometer (e.g., flow cytometer 1202 of FIG. 12).

The processing system 1300 may include a processor 1302, main memory 1306, non-volatile memory 1310, network adapter 1312, video display 1318, input/output device 1320, control device 1322 (e.g., a keyboard, pointing device, or mechanical input such as a button), drive unit 1324 that includes a storage medium 1326, or signal generation device 1330 that are communicatively connected to a bus 1316. The bus 1316 is illustrated as an abstraction that represents one or more physical buses and/or point-to-point connections that are connected by appropriate bridges, adapters, or controllers. The bus 1316, therefore, can include a system bus, Peripheral Component Interconnect (PCI) bus, PCI-Express bus, HyperTransport bus, Industry Standard Architecture (ISA) bus, Small Computer System Interface (SCSI) bus, Universal Serial Bus (USB), Inter-Integrated Circuit (I²C) bus, or bus compliant with Institute of Electrical and Electronics Engineers (IEEE) Standard 1394.

The processing system 1300 may share a similar computer processor architecture as that of a computer server, desktop computer, tablet computer, mobile phone, wearable electronic device (e.g., a watch or fitness tracker), network-connected device (e.g., a television or home assistant device), augmented or virtual reality system (e.g., a head-mounted display), or another electronic device capable of executing a set of instructions (sequential or otherwise) that specify actions to be taken by the processing system 1300.

While the main memory 1306, non-volatile memory 1310, and storage medium 1326 are shown to be a single medium, the terms “storage medium” and “machine-readable medium” should be taken to include a single medium or multiple media that stores instructions. The terms “storage medium” and “machine-readable medium” should also be taken to include any medium that is capable of storing, encoding, or carrying instructions for execution by the processing system 1300.

In general, the routines executed to implement the embodiments of the present disclosure may be implemented as part of an operating system or a specific application, component, program, object, module, or sequence of instructions (collectively referred to as “computer programs”). Computer programs typically comprise instructions (e.g., instructions 1304, 1308, 1328) set at various times in various memories and storage devices in a computing device. When read and executed by the processor 1302, the instructions may cause the processing system 1300 to perform operations to execute various aspects of the present disclosure.

While embodiments have been described in the context of fully functioning computing devices, those skilled in the art will appreciate that the various embodiments are capable of being distributed as a program product in a variety of forms. The present disclosure applies regardless of the particular type of machine- or computer-readable medium used to actually cause the distribution. Further examples of machine- and computer-readable media include recordable-type media such as volatile and non-volatile memory devices 1310, removable disks, hard disk drives, optical disks (e.g., Compact Disk Read-Only Memory (CD-ROMS) and Digital Versatile Disks (DVDs)), cloud-based storage, and transmission-type media such as digital and analog communication links.

The network adapter 1312 enables the processing system 1300 to mediate data in a network 1314 with an entity that is external to the processing system 1300 through any communication protocol that is supported by the processing system 1300 and the external entity. The network adapter 1312 can include a network adaptor card, wireless network interface card, switch, protocol converter, gateway, bridge, hub, receiver, repeater, or transceiver that includes an integrated circuit (e.g., enabling communication over Bluetooth or Wi-Fi).

### Remarks

The foregoing description of various embodiments of the claimed subject matter has been provided for the purposes of illustration and description. It is not intended to be exhaustive or to limit the claimed subject matter to the precise forms disclosed. Many modifications and variations will be apparent to one skilled in the art. Embodiments were chosen and described in order to best describe the principles of the invention and its practical applications, thereby enabling those skilled in the relevant art to understand the claimed subject matter, the various embodiments, and the various modifications that are suited to the particular uses contemplated.

Although the Detailed Description describes certain embodiments and the best mode contemplated, the technology can be practiced in many ways no matter how detailed the Detailed Description appears. Embodiments may vary considerably in their implementation details, while still being encompassed by the specification. Particular terminology used when describing certain features or aspects of various embodiments should not be taken to imply that the terminology is being redefined herein to be restricted to any specific characteristics, features, or aspects of the technology with which that terminology is associated. In general, the terms used in the following claims should not be construed to limit the technology to the specific embodiments disclosed in the specification, unless those terms are explicitly defined herein. Accordingly, the actual scope of the technology encompasses not only the disclosed embodiments, but also all equivalent ways of practicing or implementing the embodiments.

The language used in the specification has been principally selected for readability and instructional purposes. It may not have been selected to delineate or circumscribe the subject matter. It is therefore intended that the scope of the technology be limited not by this Detailed Description, but rather by any claims that issue on an application based hereon. Accordingly, the disclosure of various embodiments is intended to be illustrative, but not limiting, of the scope of the technology as set forth in the following claims.

What is claimed is:

1. A non-transitory medium with instructions stored thereon that, when executed by a processor of a computing device, cause the computing device to perform operations comprising:
obtaining a matrix of flow cytometry data that characterizes a sample containing cells labelled with fluorescent markers;
implementing a function that transforms the matrix of flow cytometry data into a vector of flow cytometry data;
providing (i) the vector of flow cytometry data and (ii) a set of labels that indicate, for each cell characterized in the vector, a pattern of immunophenotype collections to a classification model as input, so as to produce a trained classification model; and
storing the trained classification model in a data structure.

2. The non-transitory medium of claim 1, wherein the matrix of flow cytometry data includes fluorescence values over M wavelengths by N parameters, where M and N are integer values.

3. The non-transitory medium of claim 1,
wherein the matrix of flow cytometry data is one of multiple matrices of flow cytometry data that are obtained by the computing device, and
wherein the multiple matrices of flow cytometry data correspond to different samples that are known to be representative of at least two hematological diseases.

4. The non-transitory medium of claim 3,
wherein each of the multiple matrices of flow cytometry data is transformed into a corresponding vector of flow cytometry data, so as to produce multiple vectors of flow cytometry data, and
wherein the multiple vectors of flow cytometry data are provided to the classification model as input, so as to allow the classification model to learn to distinguish between the at least two hematological diseases.

5. The non-transitory medium of claim 1, wherein when applied to a new vector of flow cytometry data that corresponds to a new sample, the trained classification model produces, as output, a classification for the new sample that is based on sample-level analysis rather than cell-level analysis.

6. The non-transitory medium of claim 5, wherein the classification is representative of a proposed diagnosis for a given hematological disease that is determined based on a distribution of immunophenotype collections across the new sample.

7. The non-transitory medium of claim 1, wherein the matrix of flow cytometry data includes a first set of values for fluorescence intensity, a second set of values for forward scatter (FSC), and a third set of values for side scatter (SSC).

8. The non-transitory medium of claim 1, wherein the matrix of flow cytometry data is included in a file that is received from a flow cytometer instrument used to characterize the sample.

9. The non-transitory medium of claim 1, wherein the matrix of flow cytometry data is retrieved from a storage medium that is accessible to the computing device via a network.

10. The non-transitory medium of claim 1, wherein the function transforms the matrix of flow cytometry data into the vector of flow cytometry data through Fisher vector encoding, such that the vector is the Fisher vector representation of the flow cytometry data included in the matrix.

11. A method comprising:
receiving a Flow Cytometry Standard (FCS) file generated by a flow cytometer instrument that characterizes a sample containing cells labelled with fluorescent markers at different wavelengths;
extracting a matrix of flow cytometry data from the FCS file;
transforming the matrix of flow cytometry data into a vector of flow cytometry data; and
providing (i) the vector of flow cytometry data and (ii) a set of labels that indicate, for each cell characterized in the vector, a disease type, a disease status, or a physiological status to a classification model as input, so as to produce a trained classification model.

12. The method of claim 11,
wherein the FCS file is one of multiple FCS files received from a source, each of the multiple FCS files corresponding to a different sample,
wherein a separate vector is derived for each of the multiple FCS files based on a corresponding matrix of flow cytometry data, so as to derive multiple vectors of flow cytometry data, and
wherein the classification model is trained using the multiple vectors of flow cytometry data so that the classification model learns how to distinguish between different hematological diseases.

13. The method of claim 11, wherein said transforming comprises:
creating a mixture model based on the matrix of flow cytometry data, and
computing a gradient of the mixture model to derive the vector of flow cytometry data.

14. The method of claim 11, wherein the vector of flow cytometry data and the set of labels are included in a training dataset that further includes information regarding one or more optical parameters and one or more fluorescent marker parameters.

15. The method of claim 14, wherein the one or more optical parameters include forward scatter area (FSC-A), forward scatter width (FSC-W), forward scatter height (FSC-H), side scatter area (SSC-A), side scatter width (SSC-W), side scatter height (SSC-H), or any combination thereof.

16. A method comprising:
receiving input indicative of a request to propose diagnoses for multiple hematological diseases based on analysis of a data file;
extracting, from the data file, a matrix of flow cytometry data that characterize a sample containing cells labelled with fluorescent markers at different wavelengths;
transforming the matrix of flow cytometry data into a vector of flow cytometry data; and
providing the vector of flow cytometry data to a classification model, as input, to obtain multiple outputs,
wherein each output of the multiple outputs is representative of a proposed diagnosis for a corresponding hematological disease of the multiple hematological diseases.

17. The method of claim 16, wherein the vector of flow cytometry data is a high-dimensional vector that includes, for each cell, a value for (i) forward scatter (FSC), (ii) a FSC characteristic, (iii) side scatter (SSC), (iv) a SSC characteristic, (v) fluorescence, and (vi) a fluorescence characteristic.

18. The method of claim 17, wherein the FSC, SSC, and fluorescence characteristics are the same characteristic.

19. The method of claim 17, the FSC, SSC, and fluorescence characteristics are selected from amplitude, frequency, amplitude variation, frequency variation, time dependency, or space dependency.

20. A non-transitory medium with instructions stored thereon that, when executed by a processor of a computing device, cause the computing device to perform operations comprising:
receiving a Flow Cytometry Standard (FCS) file generated by a flow cytometer instrument that characterizes a sample containing cells labelled with fluorescent markers at different wavelengths;
extracting (i) a flow cytometry dataset and (ii) a spillover matrix from the FCS file;
performing, based on the spillover matrix, a compensation operation involving the flow cytometry dataset, so as to produce a compensated flow cytometry dataset;
implementing a function that performs doublet discrimination to ensure that each value included in the compensated flow cytometry dataset corresponds to a single cell; and
performing a normalization operation involving the compensated flow cytometry dataset, so as to produce a normalized flow cytometry dataset.

21. The non-transitory medium of claim 20, wherein the flow cytometry dataset is in the form of a matrix.

22. The non-transitory medium of claim 20, wherein the flow cytometry dataset is extracted from a data segment of the FCS file, and wherein the spillover matrix is extracted from a text segment of the FCS file.

23. The non-transitory medium of claim 20, wherein the operations further comprise:
determining, based on an analysis of the flow cytometry dataset, that compensation is necessary to improve quality of the flow cytometry dataset;
wherein the compensation operation is performed responsive to said determining.

24. The non-transitory medium of claim 20, wherein the operations further comprise:
producing a scatter plot based on forward scatter area (FSC-A) values and forward scatter height (FSC-H) values that are included in the compensated flow cytometry dataset.

25. The non-transitory medium of claim 24, wherein when implemented, the function causes the computing device to
(i) remove cells whose FSC-A value reaches its maximum value from the scatter plot,
(ii) gate a portion of the cells that remain on the scatter plot, and
(iii) calculate a coefficient of determination between the gated portion of cells.

26. The non-transitory medium of claim 25, wherein when implemented, the function further causes the computing device to
(iv) determine whether the coefficient of determination exceeds a threshold, and
(v) return data from the compensated flow cytometry dataset for the gated portion of cells responsive to a determination that the coefficient of determination exceeds the threshold.

27. The non-transitory medium of claim 25, wherein when implemented, the function further causes the computing device to
(iv) determine whether the coefficient of determination exceeds a second threshold, and
(v) perform steps (ii) and (iii) repeatedly with the gated portion of cells decreasing by a predetermined amount each time responsive to a determination that the coefficient of determination does not exceed the predetermined threshold.

28. The non-transitory medium of claim 27, wherein steps (ii) and (iii) are performed repeatedly with the gating fraction decreasing by the predetermined amount each time until the coefficient of determination exceeds the threshold.

29. The non-transitory medium of claim 20, wherein the flow cytometry dataset includes values for multiple parameters.

30. The non-transitory medium of claim 29, wherein the multiple parameters include one or more optical parameters and one or more fluorescent marker parameters.

31. The non-transitory medium of claim 29, wherein the normalization operation involves:
aggregating values belonging to each parameter of the multiple parameters as a unique feature dimension,
resampling the unique feature dimensions to a same sample size to ensure that each parameter of the multiple parameters has the same number of values, and
normalizing the unique feature dimensions so that the values are on a similar scale.

32. The non-transitory medium of claim 31, wherein said normalizing involves implementing a z-score normalization technique.

33. A method comprising:
receiving a Flow Cytometry Standard (FCS) file generated by a flow cytometer instrument that characterizes a sample containing cells labelled with fluorescent markers at different wavelengths;
extracting (i) a flow cytometry data matrix from a data segment of the FCS file and (ii) a spillover matrix from a text segment of the FCS file;
performing, based on the spillover matrix, a compensation operation involving the flow cytometry data matrix, so as to produce a compensated flow cytometry data matrix;
implementing a function that performs doublet discrimination to ensure that each value included in the compensated flow cytometry data matrix corresponds to a single cell;
performing a normalization operation involving the compensated flow cytometry data matrix, so as to produce a normalized flow cytometry data matrix; and
storing the normalized flow cytometry data matrix in a memory.

34. The method of claim 33, further comprising:
generating a visual indicium of values in the normalized flow cytometry data matrix; and
causing display of the visual indicium on an interface for review by an individual.

35. The method of claim 34, wherein the visual indicium is a report that includes analyses of the values in the normalized flow cytometry data.
